(19) United States
(12) Patent Application Publication
(10) Pub. No.: US 2022/0211624 A1
(43) Pub. Date: Jul. 7, 2022

(54) FUSION PROTEIN NANODISK COMPOSITIONS AND METHODS OF TREATMENT

(71) Applicants: Washington University, St. Louis, MO (US); The Brigham and Women's Hospital, Inc., Boston, MA (US)

(72) Inventors: Ali Javaheri, St. Louis, MO (US); Mahmoud Nasr, Boston, MA (US)

(73) Assignees: Washington University, St. Louis, MO (US); The Brigham and Women's Hospital, Inc., Boston, MA (US)

(21) Appl. No.: 17/570,104

(22) Filed: Jan. 6, 2022

Related U.S. Application Data
(60) Provisional application No. 63/134,447, filed on Jan. 6, 2021.

(51) Int. Cl. A61K 9/127; A61K 38/17; A61P 9/04

(52) U.S. Cl. CPC A61K 9/1274; A61P 9/04; A61K 38/1709

(57) ABSTRACT

Fusion protein nanodisk compositions and methods of treating a variety of disorders by administration of the fusion protein nanodisk compositions to a patient in need are disclosed. The fusion protein nanodisks provide for the combined delivery of two different apolipoproteins to a subject in need. Fusion protein nanodiscs may include a phospholipid bilayer encompassed by a fusion membrane scaffold protein. The fusion membrane scaffold protein may include two different amphipathic alpha-helical proteins, such as apolipoproteins. Methods for treating a disorder by administering a therapeutic amount of the fusion protein nanodisc described above are also disclosed.

### CROSS-REFERENCE TO RELATED APPLICATIONS

This application claims priority from U.S. Provisional Application Ser. No. 63/134,447 filed on Jan. 6, 2021, which is incorporated herein by reference in its entirety.

### STATEMENT REGARDING FEDERALLY SPONSORED RESEARCH OR DEVELOPMENT

Not Applicable.

### MATERIAL INCORPORATED-BY-REFERENCE

The Sequence Listing, which is a part of the present disclosure, includes a computer-readable form comprising nucleotide and/or amino acid sequences of the present invention. The subject matter of the Sequence Listing is incorporated herein by reference in its entirety.

### FIELD OF THE INVENTION

The present disclosure generally relates to compositions and methods for treating disorders impacted by HDL-mediated physiological processes.

### BACKGROUND OF THE INVENTION

Apolipoprotein A-I (APOA-I) and apolipoprotein M (APOM) have both demonstrated the potential to treat or prevent heart disease, heart failure (and its subtypes), ischemic injury in various tissues (heart, liver, kidney, brain), sepsis and its consequences, various cancers, and autoimmune disease. Reconstituted APOA-I therapies exist, but no studies of APOM have occurred in humans.

### SUMMARY OF THE INVENTION

Among the various aspects of the present disclosure is the provision of fusion protein nanodisk compositions and methods of treating a variety of disorders by administration of the fusion protein nanodisk compositions to a patient in need. In one aspect, the fusion protein nanodisks enable the combined delivery of apolipoproteins apoA1 and apoM which have demonstrated efficacy individually, but for which the combined effect remains to be characterized.

One aspect of the present disclosure provides for fusion protein nanodiscs that include a phospholipid bilayer encompassed by a fusion membrane scaffold protein. The fusion membrane scaffold protein includes two molecules comprising two different amphipathic alpha-helical proteins.

In some embodiments, the two different amphipathic alpha-helical proteins are selected independently from a group of apolipoproteins consisting of apolipoprotein A-I (apoA1), apolipoprotein A-IV (apoA4), apolipoprotein B (apoB), apolipoprotein C-III (apoC3), apolipoprotein D (apoD), apolipoprotein E (apoE), apolipoprotein F (apoF), and apolipoprotein M (apoM). In one embodiment, the two different amphipathic alpha-helical proteins are apolipoprotein A-I (apoA1) and apolipoprotein M (apoM). In some embodiments, the two different amphipathic alpha-helical proteins comprise at least portions of apolipoprotein A-I (apoA1) and apolipoprotein M (apoM). In some embodiments, the fusion membrane scaffold protein comprises an amino acid sequence comprising SEQ ID NO 1, portions thereof, or variants thereof.

Another aspect of the present disclosure provides for methods for treating a disorder in a patient in need by administering a therapeutic amount of a fusion protein nanodisc comprising a phospholipid bilayer and a fusion membrane scaffold protein comprising two molecules comprising two different amphipathic alpha-helical proteins, wherein the phospholipid bilayer is encompassed by the fusion membrane scaffold protein.

In some embodiments, the two different amphipathic alpha-helical proteins are selected independently from a group of apolipoproteins consisting of apolipoprotein A-I (apoA1), apolipoprotein A-IV (apoA4), apolipoprotein B (apoB), apolipoprotein C-III (apoC3), apolipoprotein D (apoD), apolipoprotein E (apoE), apolipoprotein F (apoF), and apolipoprotein M (apoM). In one embodiment, the two different amphipathic alpha-helical proteins are apolipoprotein A-I (apoA1) and apolipoprotein M (apoM). In some embodiments, the two different amphipathic alpha-helical proteins comprise at least portions of apolipoprotein A-I (apoA1) and apolipoprotein M (apoM). In some embodiments, the fusion membrane scaffold protein comprises an amino acid sequence comprising SEQ ID NO 1, portions thereof, or variants thereof.

In some embodiments, the disorder includes, but is not limited to heart disease, heart failure, ischemic injury in heart tissues, liver tissues, kidney tissues, and brain tissues, sepsis, cancers, and autoimmune diseases. In some embodiments, the disorder is chemotherapy-related cardiotoxicity and heart failure. In some embodiments, administering the therapeutic amount of the fusion protein nanodisc prevents the chemotherapy-related cardiotoxicity and heart failure without reducing the efficacy of the chemotherapy.

Other objects and features will be in part apparent and in part pointed out hereinafter.

### DESCRIPTION OF THE DRAWINGS

The following drawings illustrate various aspects of the disclosure.

FIG. 1 is a previously published graph summarizing standardized mortality ratios (SMRs) for various cancer patients with both a risk of death of ≤30% from cancer and a risk of mortality of ≥20% from heart disease, binned by follow-up time.

FIG. 2A is a previously published pie chart summarizing a population of patients admitted to hospitals for various types of heart failure: heart failure with preserved ejection fraction (HFpEF), heart failure with reduced ejection fraction (HFrEF), and heart failure with borderline ejection fraction (HFbEF).

FIG. 2B is a previously published graph summarizing cumulative mortality of the patient populations of FIG. 2A.

FIG. 2C is a previously published table summarizing 5-year outcomes of the patient populations of FIG. 2A

FIG. 3 is a previously published flow chart summarizing results of clinical studies of treatments for chemotherapy-related cardiotoxicity and heart failure.

FIG. 4 is a flow chart summarizing an idealized clinical trial design for a therapy administered to prevent cardiotoxicity without interfering with the effects of chemotherapy against cancer.

FIG. 5 is a schematic diagram illustrating the role of HDL function in the modulation of various pathophysiologies associated with heart failure.

FIG. 6A is a previously published schematic diagram illustrating the mediation of direct cardiac effects, such as myocardial ischemia/reperfusion injury, by HDL and S1P via apolipoprotein M (APOM) and sphingosine-1-phosphate (S1P).

FIG. 6B is a previously published bar graph summarizing the effects of human LDL (100 μg/g body weight), human HDL (10 and 100 μg/g body weight), and phosphate-buffered saline vehicle (PBS) injected intravenously 30 minutes before myocardial ischemia on infarct size/area at risk (AAR).

FIG. 6C is a previously published bar graph summarizing the effects of S1P (3.8, 19, and 38 ng/g body weight), human HDL (10 and 100 μg/g body weight), and 1% bovine serum albumin vehicle (BSA) injected intravenously 30 minutes before myocardial ischemia on infarct size/area at risk (AAR).

FIG. 7 is a survival probability graph comparing the survival of heart failure patients grouped by detected APOM levels.

FIG. 8A is a timeline summarizing the administration of APOM during a course of doxorubicin treatment of a population of cancer patients.

FIG. 8B is a graph comparing changes in ejection fraction (EF) of the patients treated according to the timeline of FIG. 8A to changes in EF of patients receiving doxorubicin treatment only (control).

FIG. 9A is a graph comparing the survival of APOM^{TG} mice (APOM overexpression) and control littermates in an APL model.

FIG. 9B is a graph comparing the survival of the control mice of FIG. 1A (Control (vehicle)), control mice treated with doxorubicin (Control+Dox), and the APOM^{TG} mice treated with doxorubicin (APOM^{TG}+Dox).

FIG. 9C is a graph comparing left ventricular mass (LV mass), an indicator of doxorubicin cardiotoxicity, of the doxorubicin-treated control mice and APOM^{TG} mice of FIG. 9B.

FIG. 10A is a previously published diagram showing a co-crystal structure of S1P bound to APOM, with three residues (Arg98, Trp100, and Arg116) that contact the phosphate head group region of S1P (red and green) labeled in yellow.

FIG. 10B is a previously published diagram of S1P bound to APOM as illustrated in FIG. 10A, showing a space-filling model of the head group region of S1P in the APOM molecule.

FIG. 100 contains previously published images of ApoM-Fc and ApoM-Fc-TM fusion proteins in a conditioned medium of HEK293 or Sf9 cells separated by nonreducing or reducing 10% SDS-polyacrylamide gel electrophoresis (PAGE) and detected by anti-ApoM antibodies.

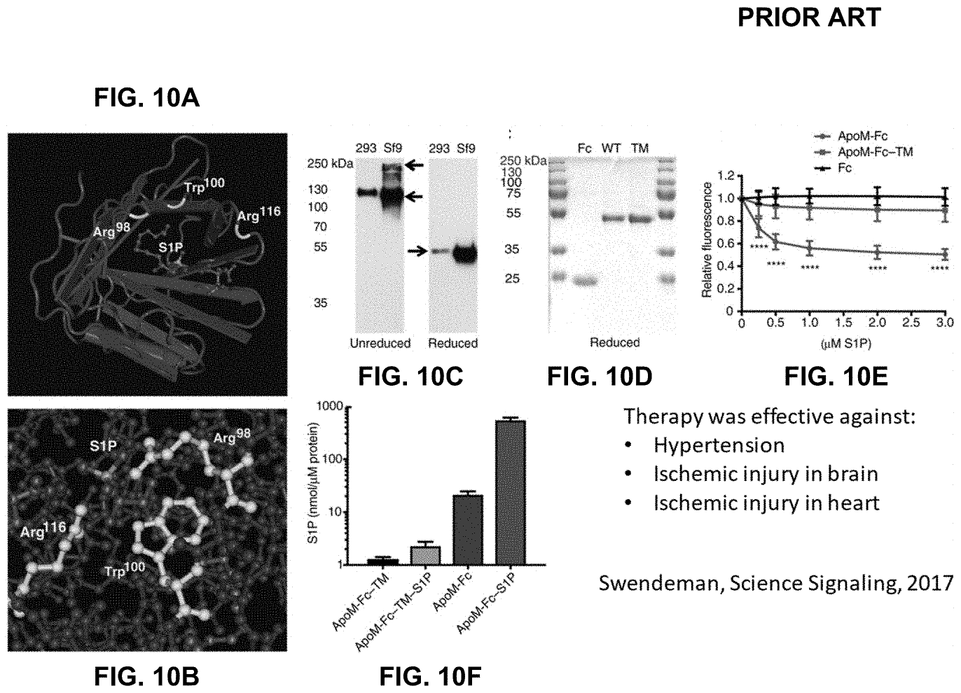

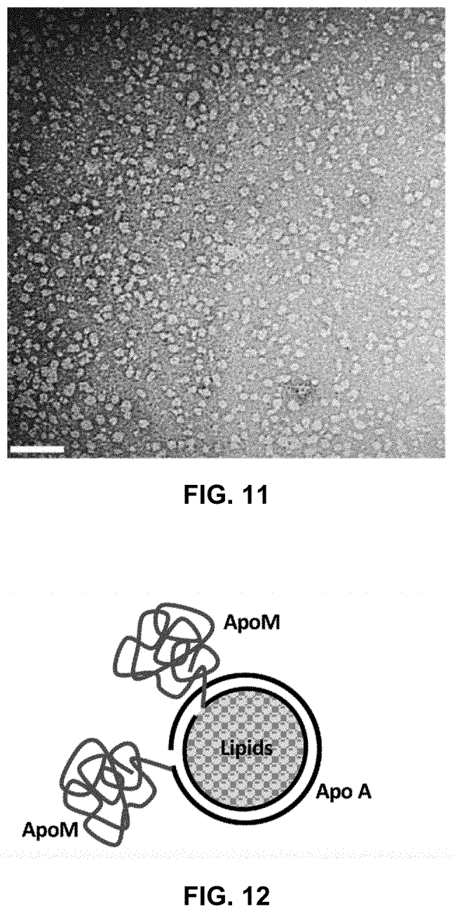

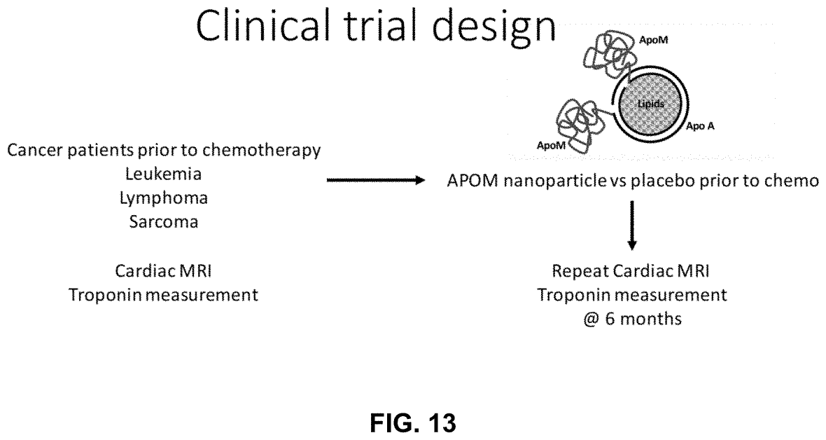

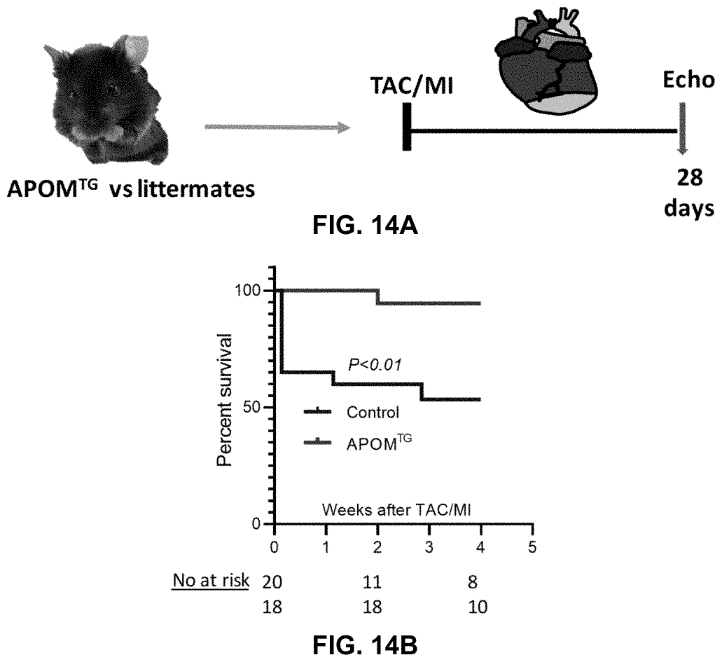

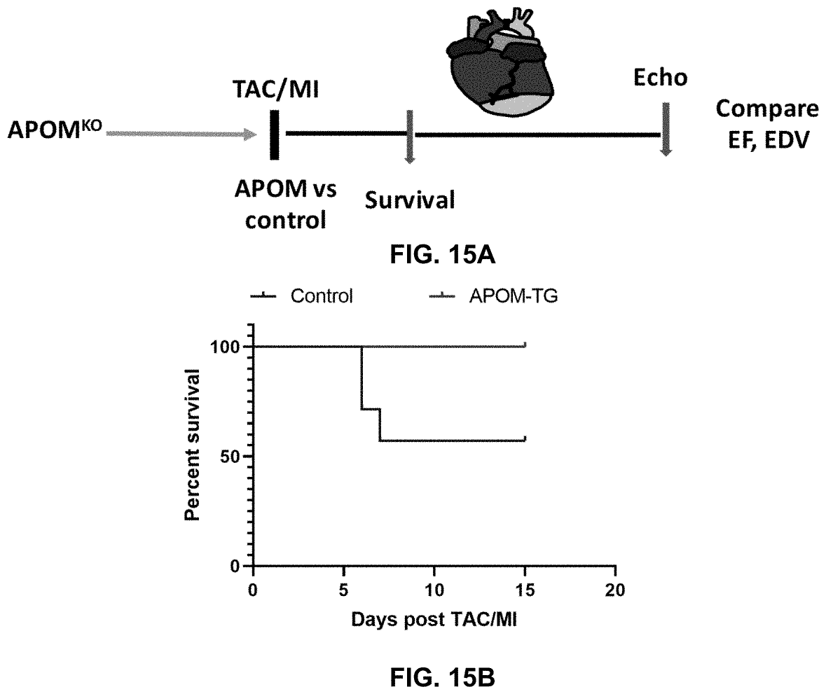

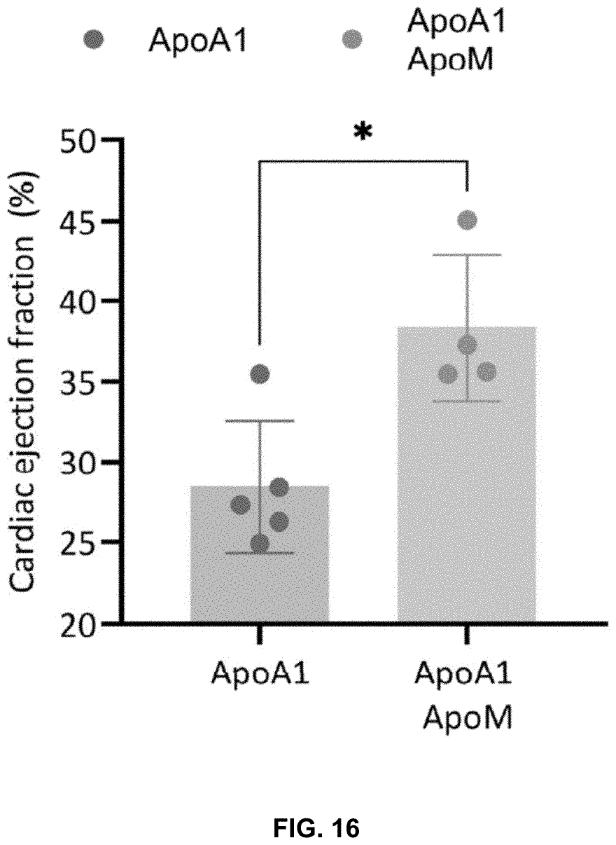

FIG. 10D is a previously published image of a gel containing Sf9-derived purified proteins (4 mg) analyzed by reducing 10% SDS-PAGE and stained with Coomassie Brilliant Blue, where WT denotes wild type.

FIG. 10E is a previously published graph comparing purified IgG1-Fc (Fc), ApoM-Fc, and ApoM-Fc-TM (TM) analyzed for 51P binding by fluorescence spectrofluorimetry; (n=4 independent experiments; mean±SD). ****P<0.001, Student's t-test and two-way analysis of variance (ANOVA) followed by Dunnett's posttest comparing ApoM-Fc or ApoM-Fc-TM to Fc alone (ApoM-Fc and ApoM-Fc-TM).

FIG. 10F is a previously published bar graph comparing sphingolipid concentrations detected by electrospray ionization-MS/MS of purified ApoM-Fc and ApoM-Fc-TM (5 mM) incubated with or without S1P for 24 to 48 hours and purified by gel filtration chromatography.

FIG. 11 is a micrograph showing nanodiscs containing APOM and APOA (APO-AM nanodiscs) in accordance with one aspect of the disclosure. Scale bar=100 nm.

FIG. 12 is a schematic illustration of the APO-AM nanodiscs of FIG. 11. Two copies of the ApoA1 lipid-binding domain (ApoA) are fused to ApoM make one nanodisc.

FIG. 13 is a schematic diagram illustrating a design of a clinical trial for the APO-AM nanodiscs of FIG. 11 administered to prevent cardiotoxicity associated with chemotherapy administered against cancer.

FIG. 14A is a timeline illustrating the treatments administered to APOM^{TG} mice (APOM overexpression) and control littermates in a TAC/MI model.

FIG. 14B is a graph comparing the survival of APOM^{TG} mice (APOM overexpression) and control littermates in the TAC/MI model treated as illustrated in FIG. 14A.

FIG. 15A is a timeline illustrating the APOM knockout treatments administered to APOM^{TG} mice (APOM overexpression) and control littermates in a TAC/MI model.

FIG. 15B is a graph comparing the survival of APOM^{TG} mice (APOM overexpression) and control littermates in the TAC/MI model treated as illustrated in FIG. 15A.

FIG. 16 is a graph comparing the cardiac ejection fraction in mice overexpressing ApoA1 versus mice overexpressing ApoA1 and ApoM following an ischemia-reperfusion induced myocardial infarction treatment.

Those of skill in the art will understand that the drawings, described below, are for illustrative purposes only. The drawings are not intended to limit the scope of the present teachings in any way.

### DETAILED DESCRIPTION OF THE INVENTION

The present disclosure is based, at least in part, on the discovery that administration of a combination of apolipoprotein A-I (APOA-1) and apolipoprotein M (APOM) may potentially treat or prevent a variety of disorders, including, but not limited to, heart disease, heart failure (and its subtypes), ischemic injury in various tissues (heart, liver, kidney, brain), sepsis and its consequences, various cancers, and autoimmune disease.

One aspect of the present disclosure provides a fusion protein nanodisc. The fusion protein nanodiscs, described in further detail herein, comprise discoidal, nanoscale phospholipid bilayers encompassed by a fusion protein comprising at least two membrane scaffold proteins (MSPs). In various aspects, each of the at least two MSPs is an apolipoprotein. Non-limiting examples of apolipoprotein include apolipoprotein A-I (apoA1), apolipoprotein A-IV (apoA4), apolipoprotein B (apoB), apolipoprotein C-III (apoC3), apolipoprotein D (apoD), apolipoprotein E (apoE), apolipoprotein F (apoF), and apolipoprotein M (apoM). In one aspect, the fusion protein comprises apoA1 and apoM, described herein as an APOA-M fusion protein. In one aspect, the fusion protein nanodisc is an APOA-M fusion protein comprising the discoidal, nanoscale phospholipid bilayers encompassed by the APOA-M fusion protein.

Various other aspects of the disclosure provide a method of treating a disorder by administering the APOA-M fusion protein to a patient in need. As described above, non-limiting examples of disorders that may be treated using the disclosed method include heart disease, heart failure (and its subtypes), ischemic injury in various tissues (heart, liver, kidney, brain), sepsis and its consequences, various cancers, and autoimmune disease.

One other aspect of the disclosure provides a method of preventing chemotherapy-related cardiotoxicity and heart failure by administering the APOA-M fusion protein nanoparticles to a patient in need. Without being limited to any particular theory, administering the APOA-M fusion protein may potentially leverage the benefits of both APOA-I and APOM proteins, as well as leverage the benefits of associated lipids to treat or prevent a variety of diseases described above.

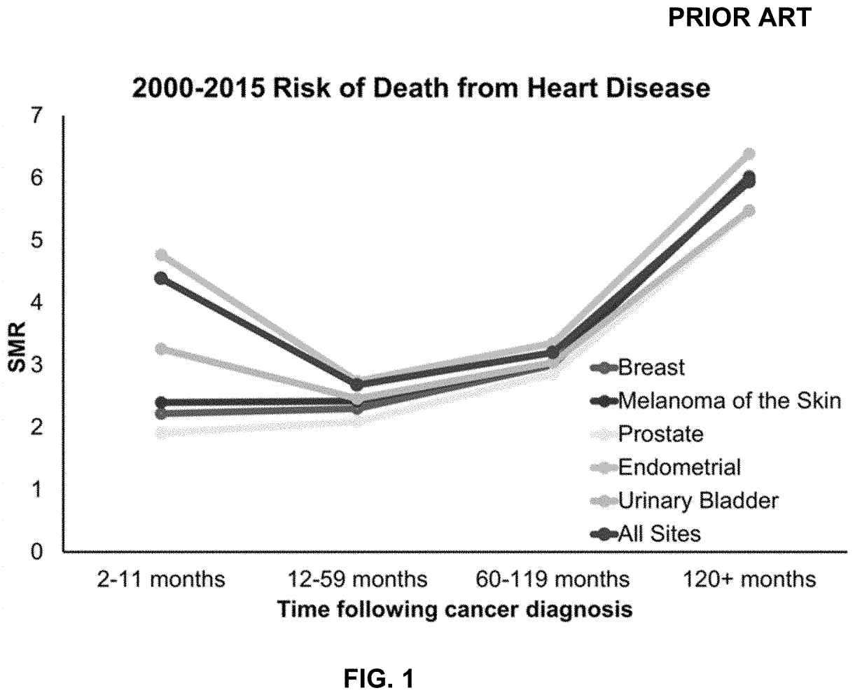

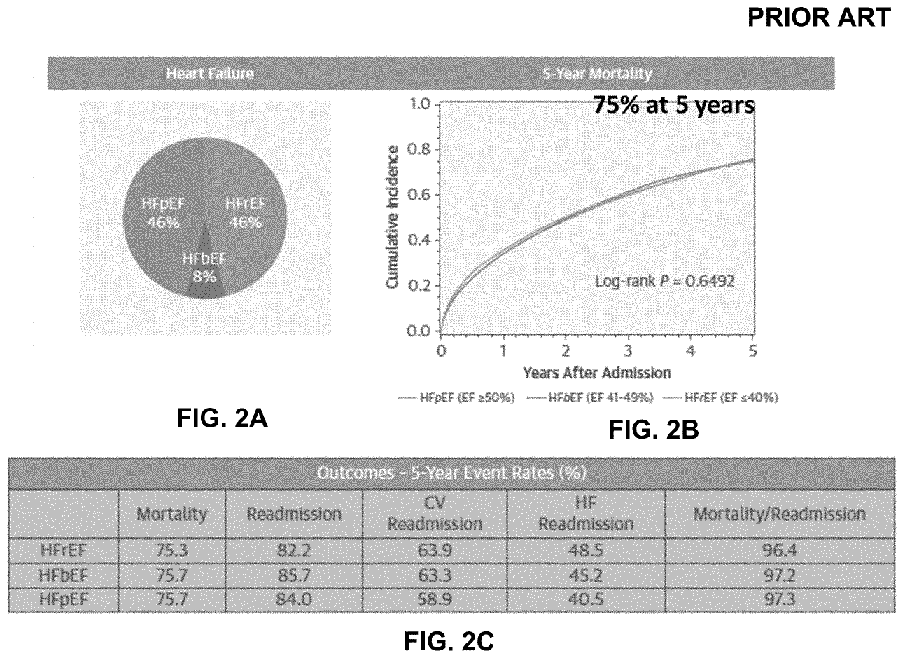

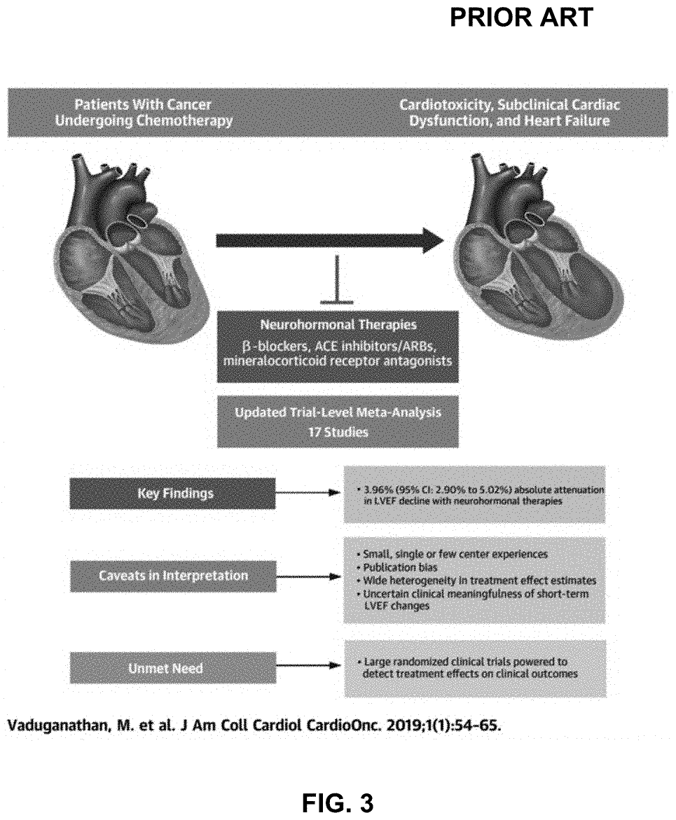

Without being limited to any particular theory, cancer patients are at elevated risk of death by heart failure likely due to the effects of cardiotoxicity associated with chemotherapy administered to the patients, as illustrated in FIG. 1. In general, heart failure has a very high mortality rate, as illustrated in FIGS. 2A, 2B, and 2C. Previous methods of preventing chemotherapy-associated cardiotoxicity by other means, such as neurohormonal therapies have had little success, as illustrated in FIG. 3.

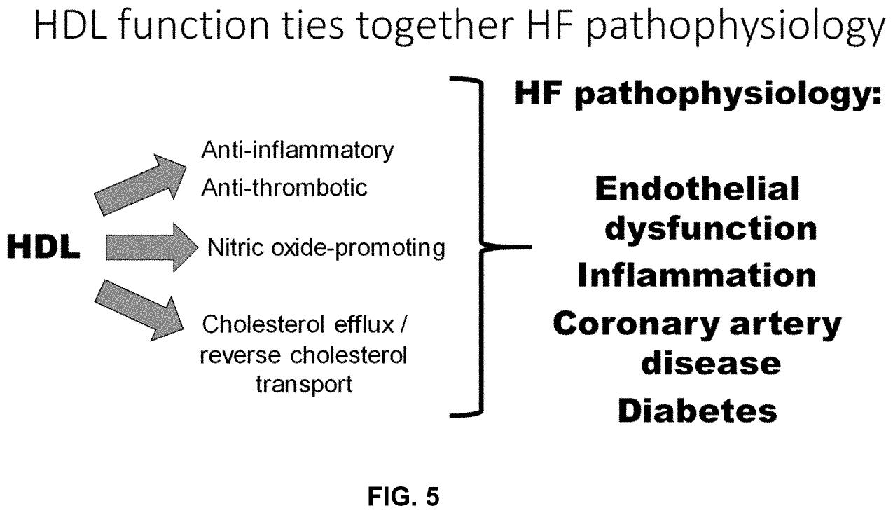

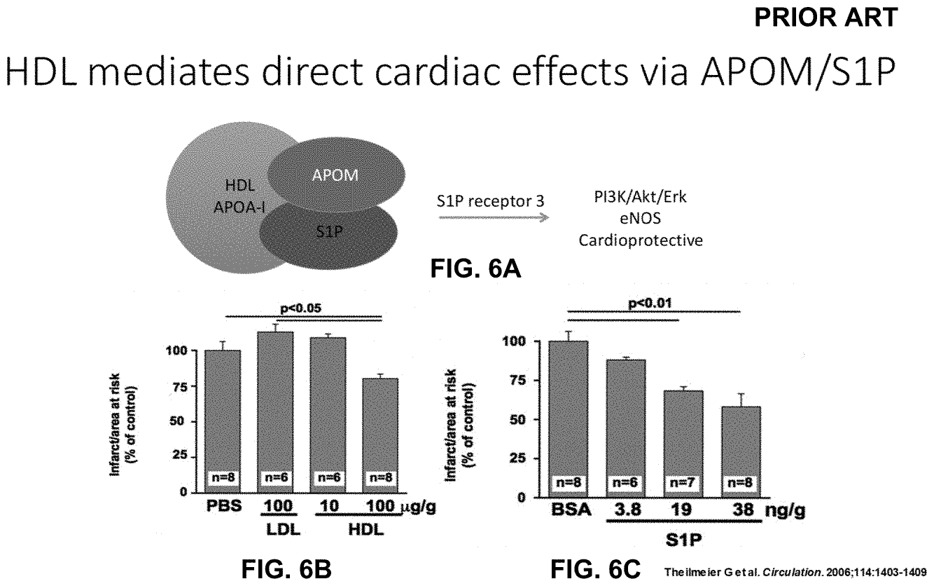

Without being limited to any particular theory, high-density lipoproteins (HDLs) are implicated within a variety of pathophysiologies associated with heart failure, as illustrated in FIG. 5. In one previously published study, high-density lipoprotein (HDL), which includes apoA1 and apoM as apolipoprotein components, was demonstrated to mediate direct cardiac effects in association with the blood-borne lipid mediator sphingosine-1-phosphate (S1P), as illustrated in FIGS. 6A, 6B, and 6C.

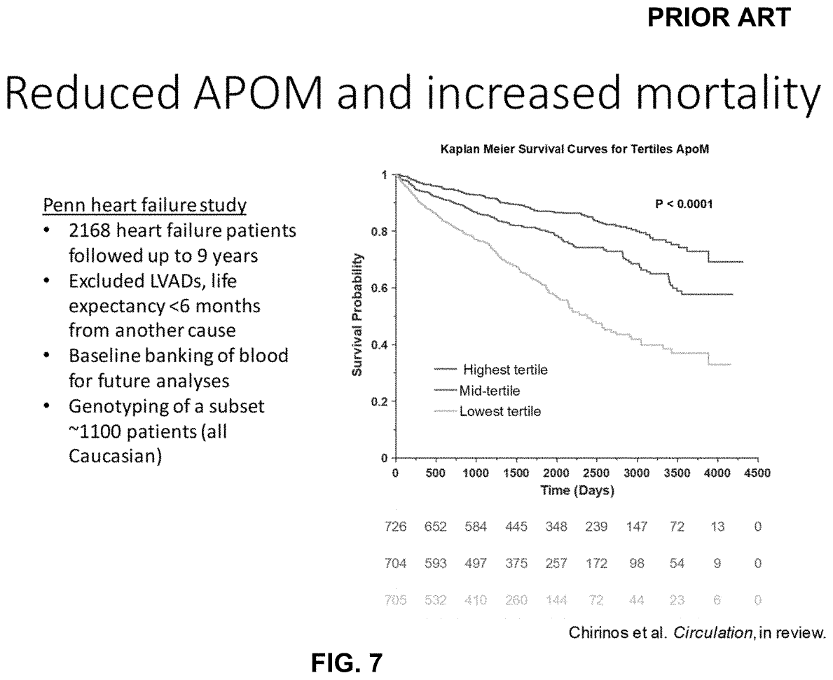

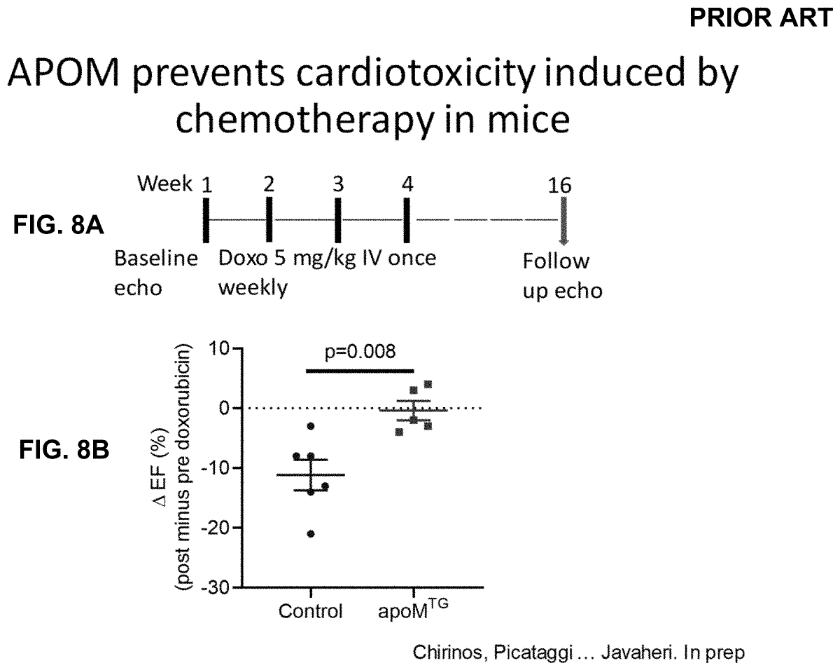

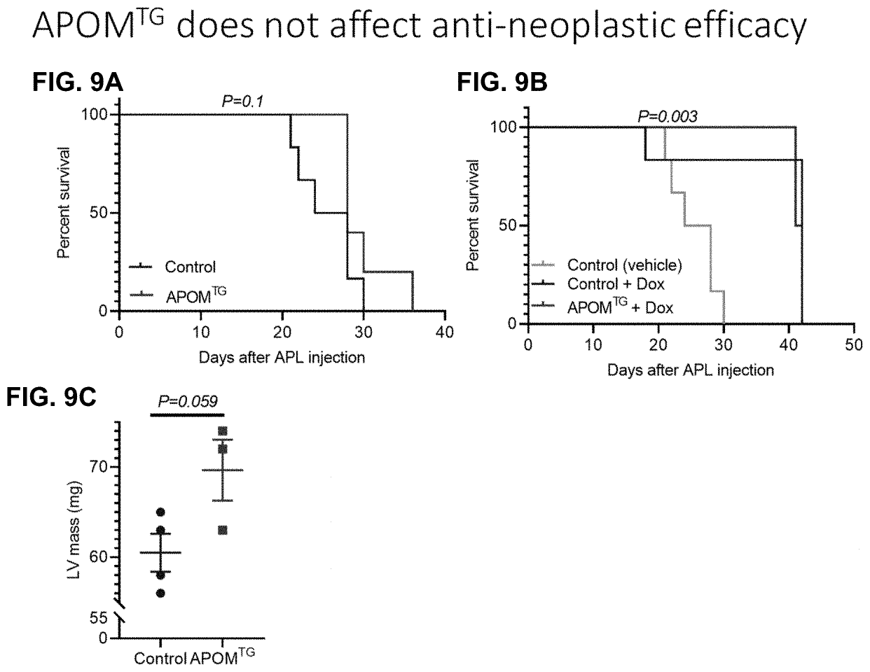

Reduced levels of apoM, one component of HDLs and other lipoproteins, have been associated with increased mortality in heart failure patients, as illustrated in FIG. 7. Enhanced apoM expression in APOM^{TG} mice (mice treated with apoM transgene for enhanced apoM expression) has been demonstrated to reduce chemotherapy-associated cardiotoxicity (see FIGS. 8A and 8B) without affecting the efficacy of chemotherapy (see FIGS. 9A, 9B, and 9C). Exogenously administered apoM composition (ApoM-Fc) was demonstrated to ameliorate a number of pathophysiologies such as hypertension and ischemic injury in the brain and heart, as illustrated in FIGS. 10A, 10B, 100, 10D, 10E, and 10F.

In one aspect, illustrated schematically in FIG. 12, the fusion protein nanodiscs comprise discoidal, nanoscale phospholipid bilayers encompassed by a membrane scaffold protein (MSP). “Membrane scaffold protein”, as used herein, refers to two molecules of amphipathic alpha-helical protein wrapped around the perimeter of the discoidal, nanoscale phospholipid bilayers in an anti-parallel fashion. The hydrophobic face of the membrane scaffold protein serves to sequester the hydrocarbon tails of the phospholipids away from the solvent and limits the size of the disc. In various embodiments, the membrane scaffold protein is an apolipoprotein. In various other embodiments, the membrane scaffold protein is a fusion protein comprising two or more apolipoproteins. In one aspect, the membrane scaffold protein is an APOA-M fusion protein comprising apolipoprotein A-I (APOA-I) and apolipoprotein M, as illustrated in FIG. 12. FIG. 11 is an image of the APOA-M fusion protein nanodiscs in one aspect. In one aspect, the fusion protein nanodiscs have a diameter of about 10 nm. In various aspects, the APOA-M fusion protein nanodiscs comprise at least one structure as described in U.S. Patent Application Publication No. 2019/0233501, the content of which is incorporated by reference in its entirety. Methods of producing the APOA-M fusion protein nanodiscs are also described in U.S. Patent Application Publication No. 2019/0233501, the content of which is incorporated by reference in its entirety.

In some aspects, the fusion protein is an APOA-M fusion protein comprising the amino acid sequence of SEQ ID NO 1, portions thereof, and variants thereof. In some aspects, the fusion protein includes a pair of ApoA-I binding domains fused to ApoM. In some aspects, the ApoA-I binding domains comprise SEQ ID NO 2, portions thereof, and variants thereof. In some aspects, the ApoM comprises SEQ ID NO 3, portions thereof, and variants thereof. SEQ ID NOS. 1, 2, and 3 are provided in Table 1 herein.

TABLE 1
APOA-M Fusion Protein Sequences

| SEQ | | |
|---|---|---|
| ID | | |
| NO | Protein | Sequence |
| 1 | APOA- | SIYQCPEHSQLTTLGVDGKE |
| | M fusion | FPEVHLGQWYFIAGAAPTKE |
| | protein | ELATFDPVDNIVFNMAAGSA |
| | | PMQLHLRATIRMKDGLCVPR |
| | | KWIYHLTEGSTDLRTEGRPD |
| | | MKTELFSSSCPGGIMLNETG |
| | | QGYQRFLLYNRSPHPPEKCV |
| | | EEFKSLTSCLDSKAFLLTPR |
| | | NQEACELSNNSTFSKLREQL |
| | | GPVTQEFWDNLEKETEGLRQ |
| | | EMSKDLEEVKAKVQPYLDDF |
| | | QKKWQEEMELYRQKVEPLGE |
| | | EMRDRARAHVDALRTHLAPY |
| | | SDELRQRLAARLEALKENGG |
| | | ARLAEYHAKATEHLSTLSEK |
| | | AKPALEDLRQGLLPVLESFK |
| | | VSFLSALEEYTKKLNTQ |
| 2 | ApoA-I | SIYQCPEHSQLTTLGVDGKE |
| | binding | FPEVHLGQWYFIAGAAPTKE |
| | domain | ELATFDPVDNIVFNMAAGSA |
| | | PMQLHLRATIRMKDGLCVPR |
| | | KWIYHLTEGSTDLRTEGRPD |
| | | MKTELFSSSCPGGIMLNETG |
| | | QGYQRFLLYNRSPHPPEKCV |
| | | EEFKSLTSCLDSKAFLLTPR |
| | | NQEACELSNN |
| 3 | ApoM | STFSKLREQLGPVTQEFWDN |
| | | LEKETEGLRQEMSKDLEEVK |
| | | AKVQPYLDDFQKKWQEEMEL |
| | | YRQKVEPLGEEMRDRARAHV |
| | | DALRTHLAPYSDELRQRLAA |
| | | RLEALKENGGARLAEYHAKA |
| | | TEHLSTLSEKAKPALEDLRQ |
| | | GLLPVLESFKVSFLSALEEY |
| | | TKKLNTQ |

In various aspects, the APOA-M fusion protein nanodiscs are compatible with methods of synthesizing in bulk for commercialization and may serve as a novel treatment for diseases such as acute heart failure, sepsis, etc. as listed above. In some aspects, the APOA-M fusion proteins may be produced using E. coli cells transformed with nucleotides encoding the APOA-M fusion protein (SEQ ID NO 1) as described in Example 3 below.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high-performance liquid chromatography. The term “RT”, as used herein, is intended to mean room temperature. The term “e.g.”, as used herein, is intended to denote an example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterions. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further includes a stoichiometric or non-stoichiometric amount of water bound by non-covalent intermolecular forces.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out so that the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine), Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, Tex.; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated in any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc., Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes, which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and may consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

Therapeutic Methods

Also provided is a process of treating a variety of disorders in a subject in need by the administration of a therapeutically effective amount of the APOA-M fusion protein nanodiscs described above to the subject in need. Non-limiting examples of disorders suitable for treatment using the APOA-M fusion protein nanodiscs include heart disease, heart failure (and its subtypes), ischemic injury in various tissues (heart, liver, kidney, brain), sepsis and its consequences, various cancers, and autoimmune disease. In one aspect, the administration of the APOA-M fusion protein nanodiscs may result in the prevention of chemotherapy-related cardiotoxicity and heart failure without affecting the efficacy of the chemotherapy on the subject.

Methods described herein are generally performed on a subject in need thereof. A subject in need of the therapeutic methods described herein can be a subject having, diagnosed with, suspected of having, or at risk for developing one of the disorders described above including, but not limited to, chemotherapy-related cardiotoxicity and heart failure. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be human.

Generally, a safe and effective amount of APOA-M fusion protein nanodiscs is, for example, that amount that would cause the desired therapeutic effect in a subject while minimizing undesired side effects. In various embodiments, an effective amount of APOA-M fusion protein nanodiscs described herein can substantially inhibit chemotherapy-related cardiotoxicity and heart failure, slow the progress of chemotherapy-related cardiotoxicity and heart failure, or limit the development of chemotherapy-related cardiotoxicity and heart failure.

According to the methods described herein, the administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of the APOA-M fusion protein nanodiscs can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to prevent chemotherapy-related cardiotoxicity and heart failure.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of the APOA-M fusion protein nanodiscs can occur as a single event or over a time course of treatment. For example, the APOA-M fusion protein nanodiscs can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for disorders including, but not limited to, heart disease, heart failure (and its subtypes), ischemic injury in various tissues (heart, liver, kidney, brain), sepsis and its consequences, various cancers, and autoimmune disease.

The APOA-M fusion protein nanodiscs can be administered simultaneously or sequentially with another agent, such as an antibiotic, an anti-inflammatory, a chemotherapy compound, or another agent. For example, the APOA-M fusion protein nanodiscs can be administered simultaneously with another agent, such as a chemotherapy compound. Simultaneous administration can occur through the administration of separate compositions, each containing one or more of the APOA-M fusion protein nanodiscs, an antibiotic, an anti-inflammatory, a chemotherapy compound, or another agent. Simultaneous administration can occur through the administration of one composition containing two or more of the APOA-M fusion protein nanodiscs, an antibiotic, an anti-inflammatory, a chemotherapy compound, or another agent. The APOA-M fusion protein nanodiscs can be administered sequentially with an antibiotic, an anti-inflammatory, a chemotherapy compound, or another agent. For example, the APOA-M fusion protein nanodiscs can be administered before or after administration of an antibiotic, an anti-inflammatory, a chemotherapy compound, or another agent.

Administration

Agents and compositions described herein can be administered according to methods described herein in a variety of means known to the art. The agents and composition can be used therapeutically as exogenous materials. Exogenous agents are those produced or manufactured outside of the body and administered to the body.

As discussed above, the administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres or nanodisks (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Such packaging of the components separately can, if desired, be presented in a pack or dispenser device, which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline, or other suitable sterile diluents each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audiotape, and the like. Detailed instructions may not be physically associated with the kit; instead, a user may be directed to an Internet website specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Effects of APOM Overexpression on Survival in Cardiac Events

To evaluate the effect of enhanced APOM concentrations on outcomes following cardiac events, the following experiments were conducted.

As illustrated in FIG. 14A, APOM^{TG} mice (mice with hepatocyte-specific APOM overexpression) and littermate controls were subjected to transverse aortic constriction (TAC) and myocardial infarction (MI) treatments, denoted below as TAC/MI treatments, and the survival percentages of both mice groups were recorded over five days following TAC/MI treatment. In another experiment, illustrated in FIG. 15A, APOM^{TG} mice and littermate controls were subjected to an APOM knockout treatment followed by TAC/MI treatment.

APOM^{TG} mice subjected to TAC/MI treatments demonstrated enhanced survival compared to control littermates, as illustrated in FIG. 14B. APOM^{TG} mice subjected to the APOM knockout treatment followed by TAC/MI treatment demonstrated enhanced survival compared to control littermates, as illustrated in FIG. 15B.

The results of these experiments demonstrated that enhanced APOM concentrations resulted in improved survival after cardiac events.

### Example 2: Effects of APOA Versus APOA-M Overexpression on Survival in Cardiac Events

To evaluate the effect of enhanced APOM concentrations in the presence of enhanced APOA concentrations on outcomes following cardiac events, the following experiments were conducted.

As illustrated in FIG. 14A, mice with APOA overexpression (ApoA1) and mice with overexpression of both APOA and APOM (ApoA1 ApoM) were subjected to an ischemia-reperfusion induced myocardial infarction treatment followed by an evaluation of cardiac ejection fraction. AS illustrated in FIG. 16, the ApoA1 ApoM mice exhibited significantly higher cardiac ejection fractions following the infarction treatment as compared to the ApoA1 mice.

The results of these experiments demonstrated that enhanced APOM and APOA concentrations resulted in improved outcomes after cardiac events as compared to enhanced APOA concentrations alone.

### Example 3: Production of APOA-M Fusion Protein Nanodiscs

To demonstrate the production of the APOA-M fusion protein nanodiscs described herein, the following experiments were conducted.

ApoA-M in pET-28a containing a tobacco etch virus (TEV) protease-cleavable N-terminal His6 tag was transformed into BL21-Gold (DE3) competent Escherichia coli cells (Agilent). The cell cultures were grown at 37° C. in Luria broth (LB) medium supplemented with 50 mg/ml kanamycin. Expression was induced at an OD600 of 0.6 with 1 mM IPTG, and cells were grown for another 3 hours at 37° C.

The cells were resuspended and sonicated in lysis buffer and the supernatant was loaded onto an Ni2+-NTA column. The resulting ApoAM protein was eluted then treated with TEV to cleave the N-terminal His6 tag.

To assemble the nanodiscs, purified ApoAM was incubated with various lipid mixtures. After incubation, the detergent (used to solubilize the lipids) was removed by incubation with Bio-beads SM-2 (Bio-Rad) or dialysis. The nanodisc preparations were filtered to remove the Bio-beads. The nanodisc preparations were further purified by size-exclusion chromatography while monitoring the absorbance at 280 nm on a Superdex 200 10×300 column. Fractions corresponding to the right size of nanodisc were collected and concentrated. Negative-stain EM was conducted to confirm the nanodisc formation. Briefly, 3.5 μl of nanodisc samples were adsorbed to glow-discharged, carbon-coated copper grids and stained with 0.75% (w/v) uranyl formate. All EM images were collected with a Philips CM10 electron microscope (FEI) equipped with a tungsten filament or T12 electron microscope equipped with a LaB6 filament and connected to a Gatan UltraScan CCD camera.

FIG. 11 is a representative transmission electron micrograph of the ApoAM nanodiscs produced as described above.

The results of these experiments successfully demonstrated a process for producing the ApoAM nanodiscs disclosed herein.

### Example 4: Clinical Trial of APOA-M Fusion Protein Nanodiscs

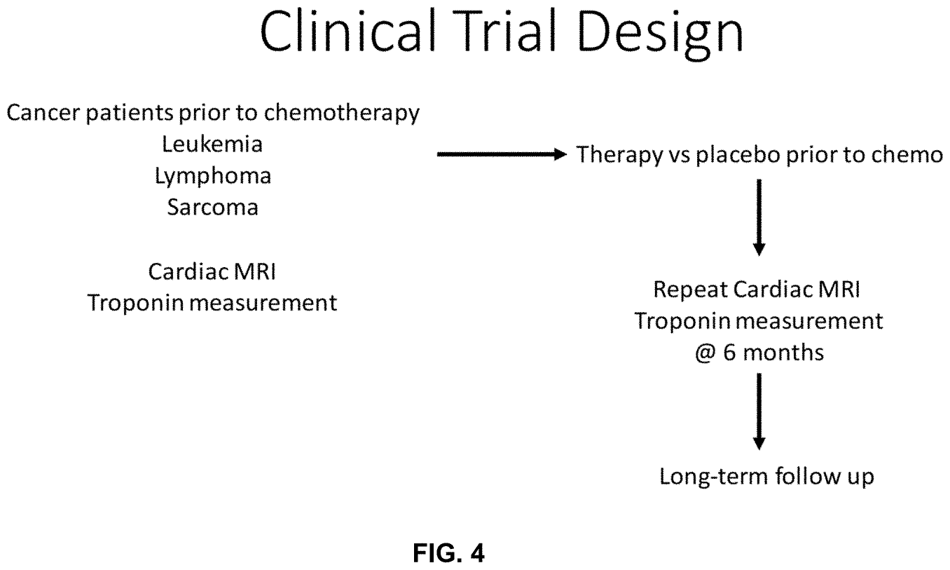

To demonstrate the efficacy of the APOA-M fusion protein nanodiscs of Example 3 at the prevention of chemotherapy-related cardiotoxicity and heart failure without affecting the efficacy of the chemotherapy on the subject, the following experiment will be conducted. A clinical trial will be designed following a general clinical trial design as illustrated schematically in FIG. 4. The clinical trial will be conducted as illustrated schematically in FIG. 13. A population of diagnosed cancer patients will be subjected to an initial cardiac evaluation that includes, but is not limited to, cardiac MRI and troponin measurements. The types of cancer diagnosed within the patient population will include, but are not limited to, leukemia, lymphoma, and sarcoma. After this initial cardiac evaluation, a portion of the patients (APOM-A treated) will be administered the APOA-M fusion protein nanodiscs (see FIG. 12), and the remaining portion of the patients (control) will be administered a placebo prior to receiving a course of chemotherapy as indicated by the diagnosed cancers of each patient. At some period after administration of the APOA-M or placebo treatments, including, but not limited to 6 months post-treatment as illustrated in FIG. 13, both APOA-M treated and control patients will be subjected to a follow-up cardiac evaluation similar to the initial cardiac evaluation described previously. One or more corresponding parameters measured in the initial and follow-up cardiac evaluations may be compared to assess the degree of chemotherapy-induced cardiotoxicity in the APOA-M treated patients relative to the control patients. In addition, one or more factors indicative of the efficacy of the chemotherapies may be measured in all patients and compared to assess whether APOA-M treatment had any impact on the efficacy of the chemotherapies.

What is claimed is:

1. A fusion protein nanodisc, comprising:
a. a phospholipid bilayer; and
b. a fusion membrane scaffold protein comprising two molecules comprising two different amphipathic alpha-helical proteins;
wherein the phospholipid bilayer is encompassed by the fusion membrane scaffold protein.

2. The fusion protein nanodisc of claim 1, wherein the two different amphipathic alpha-helical proteins are selected independently from a group of apolipoproteins consisting of apolipoprotein A-I (apoA1), apolipoprotein A-IV (apoA4), apolipoprotein B (apoB), apolipoprotein C-III (apoC3), apolipoprotein D (apoD), apolipoprotein E (apoE), apolipoprotein F (apoF), and apolipoprotein M (apoM).

3. The fusion protein nanodisc of claim 2, wherein the two different amphipathic alpha-helical proteins comprise at least portions of apolipoprotein A-I (apoA1) and apolipoprotein M (apoM).

4. The fusion protein nanodisc of claim 3, wherein the fusion membrane scaffold protein comprises an amino acid sequence comprising SEQ ID NO 1, portions thereof, or variants thereof.

5. A method for treating a disorder in a patient in need, the method comprising administering a therapeutic amount of a fusion protein nanodisc, the fusion protein nanodisc comprising a phospholipid bilayer and a fusion membrane scaffold protein comprising two molecules comprising two different amphipathic alpha-helical proteins, wherein the phospholipid bilayer is encompassed by the fusion membrane scaffold protein.

6. The method of claim 5, wherein the two different amphipathic alpha-helical proteins are selected independently from a group of apolipoproteins consisting of apolipoprotein A-I (apoA1), apolipoprotein A-IV (apoA4), apolipoprotein B (apoB), apolipoprotein C-III (apoC3), apolipoprotein D (apoD), apolipoprotein E (apoE), apolipoprotein F (apoF), and apolipoprotein M (apoM).

7. The method of claim 6, wherein the two different amphipathic alpha-helical proteins comprise at least portions of apolipoprotein A-I (apoA1) and apolipoprotein M (apoM).

8. The method of claim 7, wherein the fusion membrane scaffold protein comprises the amino acid sequence comprising SEQ ID NO 1, portions thereof, or variants thereof.

9. The method of claim 8, wherein the disorder is selected from the group consisting of heart disease, heart failure, ischemic injury in heart tissues, liver tissues, kidney tissues, and brain tissues, sepsis, cancers, and autoimmune diseases.

10. The method of claim 1, wherein the disorder is chemotherapy-related cardiotoxicity and heart failure.

11. The method of claim 10, wherein administering the therapeutic amount of the fusion protein nanodisc prevents the chemotherapy-related cardiotoxicity and heart failure without reducing the efficacy of the chemotherapy.


SEQUENCE LISTING (3 sequences)

SEQ ID NO: 1 - protein, 337 aa, Artificial Sequence
  Synthetic Sequence
SIYQCPEHSQLTTLGVDGKEFPEVHLGQWYFIAGAAPTKEELATFDPVDNIVFNMAAGSAPMQLHLRATIRMKDGLCVPRKWIYHLTEGSTDLRTEGRPDMKTELFSSSCPGGIMLNETGQGYQRFLLYNRSPHPPEKCVEEFKSLTSCLDSKAFLLTPRNQEACELSNNSTFSKLREQLGPVTQEFWDNLEKETEGLRQEMSKDLEEVKAKVQPYLDDFQKKWQEEMELYRQKVEPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 170 aa, Artificial Sequence
  Synthetic Sequence
SIYQCPEHSQLTTLGVDGKEFPEVHLGQWYFIAGAAPTKEELATFDPVDNIVFNMAAGSAPMQLHLRATIRMKDGLCVPRKWIYHLTEGSTDLRTEGRPDMKTELFSSSCPGGIMLNETGQGYQRFLLYNRSPHPPEKCVEEFKSLTSCLDSKAFLLTPRNQEACELSNN

SEQ ID NO: 3 - protein, 167 aa, Artificial Sequence
  Synthetic Sequence
STFSKLREQLGPVTQEFWDNLEKETEGLRQEMSKDLEEVKAKVQPYLDDFQKKWQEEMELYRQKVEPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ